(19) United States
(12) Patent Application Publication
(10) Pub. No.: US 2006/0148710 A1
(43) Pub. Date: Jul. 6, 2006

(54) Oligopeptides, composition and use thereof as elicitors of the natural defences of plants

(75) Inventors: Olivier Besnard, Montpellier (FR); Jean Martinez, Caux (FR); Florine Cavelier, Castelnau (FR)

Correspondence Address: YOUNG & THOMPSON, 745 SOUTH 23RD STREET, 2ND FLOOR, ARLINGTON, VA, 22202, US

(73) Assignees: DE Sangosse SA, Pont du Casse (FR); Centre National De La Recherche Scientifique, Paris Cedex 16 (FR)

(21) Appl. No.: 10/551,771

(22) PCT Filed: Apr. 2, 2003
(86) PCT No.: PCT/FR03/01021

§ 371 Date: Mar. 7, 2006

(51) Int. Cl. A01N 63/00; C07K 14/415

(52) U.S. Cl. 514/13; 530/326

(57) ABSTRACT

The invention relates to oligopeptides used as elicitors of the natural defences of plants in order to control fungal and/or bacterial and/or viral pathogens and/or crop pests, by means of foliar or root application or by injection. According to the invention, said oligopeptides are characterised in that they are obtained by organic or enzymatic synthesis, in that they are heteropolymers and/or homopolymers of protein and/or non-protein amino acids, and in that said amino acids form sequences of said polymers selected for the capacity thereof to form spiral structures.

The invention relates to oligopeptides utilized as elicitors of the natural defenses of plants against fungal and/or bacterial and/or viral pathogens and/or pests, by foliage, root or injection application, and obtained by organic or enzymatic synthesis.

The active materials of the phytosanitary preparations can have a direct action on microorganisms, which is the case of molecules inhibiting certain metabolic paths of the cells or by affecting their organization. It is generally a matter of curative treatment.

In biological pest control, the curative treatment is ensured by the use of an antagonist or parasitic agent. Similarly, there is used a microorganism which is very competitive for the colonization of the space when it is a matter of preventive treatment.

The active materials of the phytosanitary preparations can have the property of acting directly by activating the natural defense system (NDS) of the plant. This translates into the sensitivization of the plant to a possible ultimate attack by the pathogen. In this step, the genes responsible for the synthesis of defense proteins and the phyto alexines are activated, and the corresponding products are synthesized upon the first contact between the vegetable cell and its aggressor. We thus speak of a curative treatment by means of elicitors.

The defense mechanisms can be localized at the site of the attack, it is a hypersensitivity reaction (HR) which gives rise to cellular death.

They can be generalized and lead to an acquired systemic resistance (ASR).

Overall, two defense mechanisms act together to halt the propagation of the disease. On the one hand, at the site of penetration of the pathogen, the infected cells self destruct to retard its progression (hypersensitivity reaction). On the other hand, the alert signals to the neighboring cells create a zone of local acquired resistance in which numerous defense compounds accumulate. Signals are also transmitted to the entire plant, thus leading to an acquired systemic resistance (ASR).

The novel particularity of this invention is as to a new mode of action which is preventive. Thus, this new class of elicitors permits simulating an attack of pathogens in the vegetable cells. These latter will trigger a natural defense mechanism which gives rise to an ASR and this at least once, which permits preventing even more effectively the attack.

Very small quantities of elicitors suffice to sensitize the cytoplasmic membrane. Thus, the threshold of detection of the elicitors by plants can reach a value of 10⁻⁹ moles or even lower (Boller et al. 1995; Annu. Rev. Plant Physiol. Plant. Mol. Biol.)

Elicitors

Since the introduction of the term elicitor by Keen et al. (1972, Phytopathology), it has been demonstrated that several substances of various chemical structures have the property of actuating the natural defense systems (NDS) of the plants. These are particularly those of abiotic origin and which are represented by mercury, copper, aluminum ions, arachidonic, phosphoric, salicilic, fulvic and humic acids. There also exist biotic elicitors of which those best known are:

The oligosaccharides: they are part of the first elicitors to be the best characterized (Darvill and Albersheim (1984, Annu. Rev. Plant. Physiol)). They are comprised of 4 classes: oligoglucans, oligochitin, oligochitosan, oligogalacturonides, of vegetable origin (Côté et al. (1994, Plant Mol. Biol)).

Oligoglucans: the hepta-β-glucosid branched at positions (1,3-1,6) is the smallest oligoglucosid known for its elicitor action. It has been isolated from Phytophtora sojae.

Chitin: this is a linear polymer of (1,4)-N-acetyl-β-glucosamine which is found in higher mushrooms and represents the major constituent of their mycella. The soluble portion of chitin (liberated by the action of vegetable chitinase), gives rise to the lignification and the production of phytoalexins in certain plants [Pearce et al. (1982, Physiol. Plant Pathol), Ren et al. (1992, Plant Physiol)]

The oligogalacturonids: they are probably released by the degradation of homogalacturonans (pectic polysaccharides) upon an attack of the pathogen against the plant. The homogalactoronans are comprised of residues of 1,4-α-D-galactosyluronic acid and enter into the composition of the cell walls of the upper plants [Côté et al. (1994, Plant Mol. Biol)].

The enzymes:

Boland et al. (1997, FEBS Letters) have shown that the treatment of certain plants with a commercial cellulase triggers the biosynthesis of volatile products via the octadecanoic acid signal route.

Klüsener et al. (1999, FEBS Letters) have studied the interactions between cellulytic enzymes on the one hand, and an elicitor from a yeast culture (Eschscholtzia californica) on the other hand, with lipidic bilayers. They arrived at the conclusion that the elicitors can depolarize the cytoplasmic membranes, influencing the ionic flow through these latter and even disturbing their organizations in certain cases. These elicitors thus do not need to react with the intracellular proteins to give rise to defense responses in the plant. This would explain the wide spectrum of certain molecules.

The polypeptides and Proteins:

Certain free glycopeptides and/or oligosaccharides derived from glycoproteins are active in terms of elicitation [Anderson et al. (1989), Ebel et al. (1995, Can. J. Bot), Boller et al. (1995, Annu. Rev. Plant Physiol. Plant. Mol. Biol)].

In the glycoproteins of Collectotrichum lindemuthianum (Coleman et al. 1992, Physiol Mol Plant Pathol) and of Puccinia graminis f.sp. tritici (Kogel et al. 1988, Physiol Mol Plant Pathol), the glucidic portions are responsible for the elicitor activity.

A group of phytopathogenic bacteria of the Gram-negative type produce elicitor proteins of the HR in non-host plants. For example, Erwinia amylovara (Wei et al. 1992, Science; Baker et al. 1993, Plant Physiol) has the gene hrpN which codes for the production of a harpin, and Pseudomonas syringae pv. syringae (He et al. 1993, Cell) secretes a harpineₛₛ which is the product of the hrpZ gene. The protein (HrpN) from Erwinia amylovora stimulates the extracellular flow of the K⁺ cations and thus regulates the current in the cells of Arabidopsis thaliana.

A protein of 18 kDa secreted by a strain of Trichoderma virens has been isolated and characterized by Hanson et al. (2000, Phytopathology). This protein is capable of inducing biosynthesis of the products of the terpenoid types in cotton.

Benhamou et al. (2000, Plant physiology) disclose that a protein (oligandrin) of low molecular weight, from Pythium oligandrum, would induce a defense reaction in tomato plants. This reaction would limit the progression of the malady caused by the phytopathogenic agent Phytophtora parasitica.

Cryptogene is a protein secreted by the mushroom Phytophtora cryptogea (Blein et al. 1997, FEBS Letters) which serves for the transport of sterol. It is also known for its stimulating properties of the defenses of plants (Ricci et al. 1989, Eur. J. Biochem).

Oligopeptides:

The mushroom Phytophtora sojae produces a glycoprotein of which a portion (42 kDa) has the elicitor property. There has also been identified in the C-terminal portion of this glycoprotein, an oligopeptide of 13 amino acids which would also be active [(Parker et al. 1991, Mol Plant-Microbe Interact.), (Nûrnberger et al. 1994, Cell), (Sacks et al. 1995, Mol Gen Genet.)]. A specific structure and a minimum length of the sequence are essential so that this oligopeptide can keep its activity intact. Similar phenomena have been observed in the case of systemine (Pearce et al. 1993, J. Biol Chem).

Several species of Phytophtora secrete extra-cellular proteins of low molecular weight (10 kDa) called elicitins [(Ricci et al. 1992, Plant Pathol), (Kamoun et al. 1994, Appl Environ Microbiol), (Boissy et al. 1996, Structure)]. These molecules are capable of giving rise to hypersensitivity reactions as well as an acquired systemic resistance [(Ricci et al. 1989, Eur. J. Biochem, 183 (3)), (Kamoun et al. 1993, Mol Plant-Microbe Interact)].

The peptide AVR9 Cladosporium fulvum is an elicitor (Wit et al. 1997, Mol Plant-Microbe Interact) of the hypersensitivity reaction in tomato plants having the resistance gene (MM-Cf9).

Amino acids:

Siegrist et al. (2000, Physiological and Molecular Plant Pathology) have studied the capacity of certain of these amino acids to trigger the ARS in tobacco plants. Working with concentrations of 10 mM, they have observed the following results:

β-aminobutyric acid is active,

α-aminobutyric acid is less active,

γ-aminobutyric acid is totally inactive.

Specifics of the Invention

The specifics of the invention concern the use of spiral Oligopeptides.

The works of Boland et al. (2000, Angew. Chem. In. Ed.) have shown that peptaibols give rise to the biosynthesis of volatile products in certain plants. This would be due to their capacity to form ionic channels in the cellular membranes. These types of actions seem to be those which would trigger the stimulation of the strongest natural defense. They would be due particularly to localized necrosis of the touched cells, which leads to a generalized alert in the adjacent cells and tissues.

According to Pedras et al. (1997, Phytochemistry), destruxine B (cyclic oligodepsipeptide) secreted by the mushroom Alternaria brassicae during attack of the crucifers, would lead to the biosynthesis of a phytotoxin called sinalexin.

Bodo et al. (1998, Biochem. and Biophys. Acta) have studied the interaction of the peptaibols with liposomes [wide unilamellar vesicules (WUV)]. They have been able to show that the major process involved in the ionic exchange through the membrane would be the “all or nothing” mode. The formation of pores sufficiently wide is thus essential for the transition of the different ions. This is ensured by the formation of a supramolecular complex between the aggregate of 3 to 4 peptidic monomers and the lipidic molecules.

Yeo et al. (2000, Tetrahedon Letters) have shown the inhibitory activity of the peptavirines A and B against the tobacco mosaic virus. An inhibition of 74 to 79% has been observed by using concentrations of 10 μg/ml. These authors speak of a mechanism of direct action of the peptavirines in the inhibition of the pathogen, but in no case do they invoke the eliciting action of these peptaibols.

The invention relates to obtaining spiral oligopeptides by peptidic synthesis. The spiral structures of these peptides are disposed within cellular membranes. When several of these molecules are inserted together in the membrane, they form a channel or a pore, which changes the membrane permeability (depolarization). The originality of this invention is thus to clarify, optimize and obtain these molecular structures which permit forming these “pores” with certainty.

Synthesis of Amino Acid Polymers

A—Polymers Obtained in Mixtures

1. Obtaining Polyaminoacid-Alcohols:

The reaction between an amino alcohol derived from an amino acid and from an N-Carboxyanhydride derived from an amino acid, identical to R₁=R₃ and R₂=R₄, or different, in an organic solvent and under known stoechiometric conditions, leads to the formation of a mixture of homopolymers or heteropolymers respectively.

The process permitting obtaining this type of product can be described by the following reaction scheme:

a) From α-aminoalcohol and α-N-carboxyanhydride

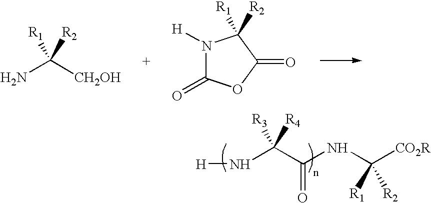

b) From α-aminoalcohol, β-aminoalcohol and β-N-carboxyanhydride (Cheng et al., 2000, Organic letters)

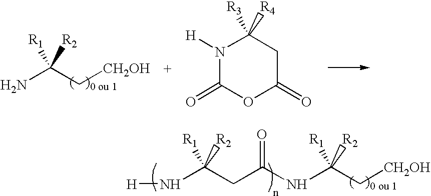

In the two cases a and b: R=H, alkyl, substituted alkyl.

According to the L or D configuration of the amino acids used:

R₁ and R₃=H, alkyl, substituted alkyl or side chain of natural amino acids or non-natural amino acids (protected in the case of functional chains): R₁ and R₃ can be identical;

R₂ and R₄=H, alkyl, substituted or side chain alkyl of natural or unnatural amino acids (protected in the case of functional chains);

n is comprised between 3 and 30.

Synthesis process:

To 20 equivalents of NCA (N-carboxyanhydride α or β) of amino acid dissolved in the selected solvent (dichloromethane, dimethylformamide, acetonitril, 20 ml of solvent per equivalent of NCA amino acid α or β), is added 1 equivalent of aminoalcohol (α or β). The reaction medium is agitated at ambient temperature for 6 to 48 hours, according to the amino acid used. The precipitate obtained is then filtered.

Examples:

| | | |
|---|---|---|
| Alaninol | R₁ = CH₃ | R₂ = H |
| NCA of alanine: | R₃ = CH₃ | R₄ = H |
| NCA of glutamic acid | R₃ = CH₂CH₂COOBzl | R₄ = H |
| NCA of valine | R₃ = CH(CH₃)₂ | R₄ = H |

2—Obtaining Polyaminoacids:

a) Starting from α-amino acid and α-N-carboxyanhydride

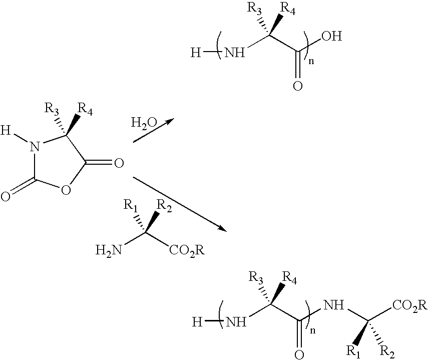

b) Starting from α-amino acid, β-amino acid and β-N-carboxyanhydride

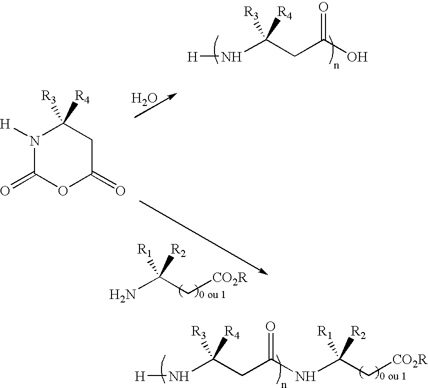

In the two cases a and b: R=H, alkyl, substituted alkyl.

According to the L or D configuration of the amino acids used:

R₁ and R₃=H, alkyl, substituted or side chain alkyl of natural or unnatural amino acids (protected in the case of functional chains). R₁ and R₃ can be identical;

R₂ and R₄=H, alkyl, substituted or side chain alkyl of natural or unnatural amino acids (protected in the case of functional chains). R₂ and R₄ can be identical;

n is comprised between 3 and 30.

Synthesis Process:

To 20 equivalents of NCA (N-carboxyanhydride α or β) amino acid dissolved in the suitable solvent (dichloromethane, dimethylformamide, acetonitrile, 20 ml of solvent per equivalent of NCA of amino acid α or β), is added 1 equivalent of water. The reaction is agitated at ambient temperature for 6 to 48 hours. The obtained precipitate is filtered.

Examples:

| | | |
|---|---|---|
| NCA of alanine: | R₃ = CH₃ | R₄ = H |
| NCA of glutamic acid | R₃ = CH₂CH₂COOBzl | R₄ = H |
| NCA of valine | R₃ = CH(CH₃)₂ | R₄ = H |

3. Obtaining Acylated Polymers:

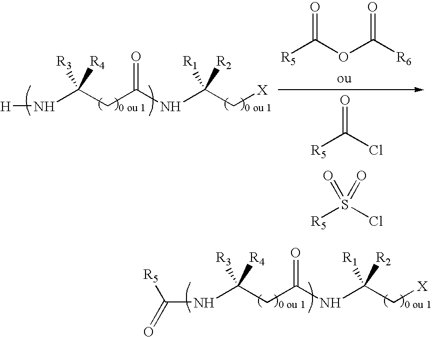

R=H, alkyl, substitute alkyl.

Ac cording to the L or D configuration of the amino acids used:

R₁ and R₃=H, alkyl, substituted or side chain alkyl of natural or unnatural amino acids (protected in the case of functional chains). R₁ and R₃ can be identical;

R₂ and R₄=H, alkyl, substituted or side chain alkyl of natural or unnatural amino acids (protected in the case of functional chains). R₂ and R₄ can be identical;

n is comprised between 3 and 30.

Process of Acylation:

To 250 mg of products to be acylated are placed in suspension in 30 ml of dimethylformamide. 15 equivalents of acylating agent (acetic anhydride, decanoic anhydride, etc . . . ) are added. The reaction is agitated at ambient temperature (from 16 to 48 hours).

After filtration, a white powdery solid is obtained.

B—Pure Polymers Obtained and Characterized

1. Polyaminoacid-Alcohols:

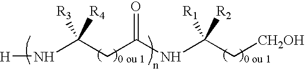

R=H, alkyl, substituted alkyl.

According to the L or D configuration of the amino acids used:

R₁ and R₃=H, alkyl, substituted or side chain alkyl of natural or non-natural amino acids (protected in the case of functional chains). R₁ and R₃ can be identical;

R₂ and R₄=H, alkyl, substituted or side chain alkyl of natural or unnatural amino acids (protected in the case of functional chains). R₂ and R₄ can be identical;

n, comprised between 3 and 20, is defined for each compound.
2. Polyamino Acids:

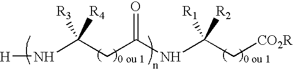

R=H, alkyl, substituted alkyl.

According to the L or D configuration of the amino acids used:

R₁ and R₃=H, alkyl, substituted or side chain alkyl of natural or unnatural amino acids (protected in the case of functional chains). R₁ and R₃ can be identical;

R₂ and R₄=H, alkyl, substituted or side chain alkyl of natural or unnatural amino acids (protected in the case of functional chains). R₂ and R₄ can be identical;

n, comprised between 3 and 20, is defined for each compound.
3. Polymer Activities:

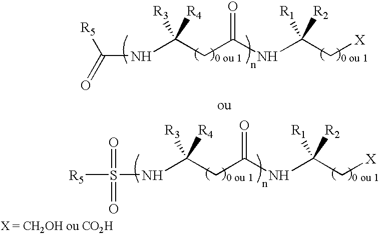

R=H, alkyl, substituted alkyl.

According to the L or D configuration of the amino acids used:

R₁ and R₃=H, alkyl, substituted or side chain alkyl of natural or non-natural amino acids (protected in the case of functional chains). R₁ and R₃ can be identical;

R₂ and R₄=H, alkyl, substituted or side chain alkyl of natural or non-natural amino acids (protected in the case of functional chains). R₂ and R₄ can be identical;

n, comprised between 3 and 30, is defined for each compound.

Example of Process for Synthesis of Pure Compounds

The synthesis of compounds 7, 8, 9, 10, 11, 12, 14 and 15 is carried out in solid phase by Fmoc strategy. The selected resin is of the 2-chlorotrityle type (Senn, 1.8 mmoles/g).

The couplings are carried out in the presence of HOBT (2.5 equivalents)/HBTU (2.5 equivalents)/DIEA (4 equivalents).

The solvent used for the introduction of the Fmoc-amino acids is dimethylformamide. The Fmoc group is cleaved by a solution of piperidine at 20% in dimethylformamide.

Grafting of Aminoalcohol:

The resin is agitated at ambient temperature in the presence of Fmoc-aminoalcohol and 6 equivalents of pyridine in a mixture constituted by dimethylformamide and dichloromethane (1/1). After 16 hours of reaction, methanol is added to the resin and the reaction mixture is agitated for 30 minutes.

After filtration, the quantity of load of the resin is determined by UV analysis.

The mass spectra ESI have been recorded on a mass spectrometer (Micromass Platform II) in the electrospray mode.

Example of Synthesis: Compound 7

Coupling-deprotection: To 1 g of resin preloaded with alaninol (quantity of substitution 0.15 mmol.g⁻¹) in suspension in DMF, is added a solution of Fmoc-Ala in the DMF then an activation solution comprised by an equimolar mixture of HBTU and HOBt at 0.5 M and 4 equivalents of DIEA in the DMF. The mixture is agitated 6 hours, then treated with a solution of piperidine at 20% in DMF. The resin is washed with a solution of dichloromethane then with a solution of ether.

Cleaving the resin: The resin is transferred from the reaction vessel to a hemolysis tube to which has been added 4-5 ml of a solution of TFA at 50% in dichloromethane. After 10 minutes under agitation, the solution is filtered and the resin washed with dichloromethane. The solvent is evaporated under vacuum.

The compounds 8-15 are prepared by repeating n times the step of coupling-deprotection.

1: Ac-Alaₙ-Ala-ol (1<n<10)

ES: [M+H]⁺: 261.0, 402.5, 471.7, 544.4, 615.1, 686.4, 757.3, 808.9

2: Ac-Alaₙ-Ala-ol (1<n<5)

ES: [M+H]⁺: 373.2, 444.5 ET 515.3

3: Ac-Alaₙ-Ala-ol (1<n<8)

ES: [M+H]⁺: 331.3, 402.2, 473.3, 544.6, 615.5, 685.3.

4: Dodecyl-Alaₙ-Ala-ol (<n<)

ES: [M+H]⁺: 328.1, 400.0, 471.8, 542.0.

5: Ac-Alaₙ-Ala-ol (1<n<9)

ES: [M+H]⁺: 189.1, 260.0, 331.1, 402.0, 472.3, 544.5, 615.7, 686.5, 757.8

6: H-Alaₙ-Ala-ol (1<n<10)

ES: [M+H]⁺: 147.0, 218.0, 288.6, 360.1, 431.1, 502.1, 573.3, 786.5.

7: H-Ala₁-Ala-ol (MW: 146)

ES: [M+H]⁺: 147.0

8: H-Ala₂-Ala-ol (MW: 217)

ES: [M+H]⁺: 218.0; [M+NA]⁺: 240.0; [2M+H]⁺ 434.8; [2M+NA]⁺: 457.4

9: H-Ala₃-Ala-ol (MW: 288)

ES: [M+H]⁺: 289.0

10: H-Ala₄-Ala-ol (MW: 359)

ES: [M+H]⁺: 360.2; [M+NA]⁺: 382.2; [2M+H]⁺: 719.7; [2M+NA]⁺: 741.5

11: H-Ala₅-Ala-ol (MW: 430)

ES: [M+H]⁺: 431.4; [M+NA]⁺: 453.5; [2M+H]⁺: 861.3; [2M+N]⁺: 883.8

12: H-Ala₆-Ala-OH (MW: 501)

ES: [M+H]⁺; 502.4; [M+N]⁺: 524.2

13; H-Alaₙ-Ala-OH (1<n<10)

ES: [M+H]⁺: 160.9; 233.8; 303.1; 374.0; 445.2; 516.2; 587.3; 658.6; 729.3; 800.8

14: H-Ala₇-Ala-OH (MW: 572)

ES: [M+H]⁺: 573.3; [M+NA]⁺: 595.4

15: H-Ala₈-Ala-OH (MW: 643)

ES: [M+H]⁺: 644.7; [M+NA]⁺: 666.5; [2M+H]⁺: 1288.1

Physiological Effects of Oligopeptides

1—Choice of Biochemical Markers on Whole Plants

a) Measurement of the Peroxydasic Acttivity (Enzyme Endogenous to Plants)

The peroxydases hold a predominant position in the resistance mechanisms of plants.

They take part in the production of active species of oxygen toxic for pathogenic agents and are implicated in the formation of the hypersensitivity reaction. They intervene also in the modification of the cell wall. There results an increase of the synthesis of the lignin and/or of the suberine (mechanical barriers). Similarly, the covalent bonds between the proteins of the cellular wall are produced. They permit the oxidation of very toxic phenols and quinones and the incorporation of the flavonoids in the walls (chemical barrier).

Given the primordial role of the peroxydases in the resistance of plants, we have used the peroxydasic activity as a marker of the resistance following treatments by elicitors.

So as to measure the peroxydasic activities, leaves are crushed in a citrate-monohydrogeno-phosphate-disodium buffer. The extract is then placed in gaiacol and oxygenated water. A rapid reaction takes place: the gaiacol is transformed into tetragaiacol. The appearance of tetragaiacol in the medium permits us to calculate the peroxydasic activity.

Measurements are carried out in this same buffer by using the gaiacol as substrate. The results are expressed in ΔDO/mn/g of fresh material.

b) Measurement of the Chitinasic Activity

So as to verify that the mechanisms of resistance are triggered, we have also measured an enzymatic activity appearing in the situations of resistance: the chitinasic activity (enzyme synthesis upon attack of the parasites, which degrades chitin, constituting the walls of the phytopathogenic mushrooms).

Colormetric reaction used: there is used an acetate buffer:

Solution of chitin+enzymatic extract+acetate buffer qsp 0.5 ml (blue at 2 mg/ml) crude 50 mM-(pH5) (100 microliters)

incubation for 30 minutes at 37° C., and under continuous agitation.

the reaction is stopped by addition of a solution of HCL 1N

colormetric or spectrophotometric measurement at 550 nm

The chitinasic activity is expressed as ΔDO/mn/g of fresh material.

Vegetable Material Used

All the plants tested are young plants obtained from seed or by slipping. Pulverization is effected of the formulations obtained from oligopeptides. These formulations contain various wetting or penetrating agents capable of carrying the active material (oligopeptides) to the cells.

Elicitor Effects of Oligopeptides

The elicitor effects of synthetic oligopeptides have been studied in several families of plants among which can be cited: zucchini, melon, cucumber, lettuce, wheat, grape.

In the Case of Zucchini

Zucchini plants aged 3 weeks have been treated with oligopeptides. The results obtained are set forth in Table I (Sheet 1/2).

According to the results of said table, it will be noted that it is the product 6 which has the strongest elicitor activity.

Admitting that the value of 100% of peroxydasic activity is represented by the product 6, the products 10 and 12 show an activity of 50%. This difference of activity could find its explanation in the diversity of the chemical structures of these products.

At different degrees, all the other products have shown an elicitor activity. The results obtained indicate that among the products tested, the free amino acids are much less active relative to the oligopeptides. Alaninol is the most active of the residues tested, followed in order by alanine, Aib and finally GABA.

These results show clearly the elicitor power of the oligopeptides.

In the Case of Grape

Grape plants aged 3 weeks have been treated with oligopeptides. The results obtained are assembled in Tables II, III (Sheet 1/2).

The correspondence between structure and code of the synthesized products is set forth in Table IV (Sheet 2/2).

Effect of Anti-Pathogenic Protection of the Oligopeptides

### EXAMPLE 1

### Example Against Melon Fusariose

The pulverization of oligopeptides formulated in melon plants of seven days and inoculated with fusarium oxysporum fsp melonis four days before, permits obtaining protection against the pathogen. In parallel, a quantity of specimen plants had its leaves sprayed with water.

Ten days after inoculation, the symptoms appeared only in the inoculated plants treated with water. Three weeks after infection, these young infected plants dried out and died.

The plants inoculated and treated with the oligopeptides have no symptoms before six weeks and continued thereafter to develop normally. These results show clearly the elicitor power of these oligopeptides on the resistance of melon plants relative to fusarium sp.

### EXAMPLE 2

### Effect Against Aerial Maladies of Melon

By spraying oligopeptides on young melon plants, there is observed an effect similar to protection against oidium.

Five days after inoculation of the pathogen, the symptoms appeared only on plants treated with water. Those treated with oligopeptides had little or no symptoms and continued to develop normally three weeks after inoculation.

According to the basic characteristics of the invention, the oliopeptides used as elicitors:

are obtained by organic or enzymatic synthesis;

have the particularity of being hetero and/or homopolymers of amino acids, protein or non-protein, constituting sequences of said polymers which are selected for their property to form structures of the spiral types or in the form of β layers.

The composition according to the invention can:

comprise at least one oligopeptide comprising at least one amino acid of the protein type, natural and/or synthetic, and/or non-protein, natural and/or synethetic;

be present either in the form of liquid, particularly an aqueous solution, or in solid form, particularly a powder, granules or by cladding seeds.

The oligopeptides used can be incorporated in a vehicle used in agriculture of the wetting and penetrating type.

The use of oligopeptides, according to the invention, has the effect of reducing, when they are applied:

in cereals, particularly wheat, corn and rice, the attack of oidiums, septorioses, molds, fusarioses, pyricularioses and bacterial and viral maladies;

in fruit trees, particularly pear trees and apple trees, the attack of oidiums, tavelures, moniloses, bacterial and viral maladies such as “Sharka”;

in grape, the attack of oidium, of mildew, of Botrytis, of maladies of the wood, of telluric and viral maladies such as “Short-Setting”;

in lawns and in horticulture, the attack of pythiaces, mushrooms with sclerotes, fusarioses, oidiums, bacterial and viral maladies;

in oil producers, particularly soy, sunflower, melon, carrot, cauliflower and potato, the attack of oidiums, mildews, phythiaces (Phytophtora, Pythium), mushrooms with sclerotes (Rhizoctonia, Sclerotinia, Pyrenocheta), vascular mushrooms (Fusarium, Verticillium), bacterial and viral maladies.

TABLE I

| | | Percent of |
|---|---|---|
| | Peroxydasic | peroxydasic |
| | activity | activity relative |
| Product (3.5 mg/L) | (ΔDO/min/aMF) | to the product |
| 7 | 99.16 | 28.49 |
| 8 | 94.6 | 27.18 |
| 9 | 112 | 32.18 |
| 10 | 194 | 55.74 |
| 11 | 102 | 29.31 |
| 12 | 175 | 50.28 |
| 6 | 348 | 100 |
| Alininol | 69.46 | 19.96 |
| Alanine | 65.78 | 18.9 |
| Aib | 53.1 | 15.26 |
| GABA | 49.1 | 14.11 |
| Wetting only | 73.5 | 13.95 |
| Untreated control | 47.77 | 13.72 |

TABLE II

| | | Percent of |
|---|---|---|
| | | peroxydasic |
| | | activity relative |
| | Peroxydasic | to the most active |
| Product | activity | product |
| TNT | 164.59 | 71.68 |
| 6 | 187.5 | 81.66 |
| 13 | 136.6 | 59.49 |
| Reference product | 229.59 | 100 |

TABLE III

| | | Percent of |
|---|---|---|
| | | chitinasic |
| | | activity relative |
| | Chitinasic | to the most active |
| Product | activity | product |
| TNT | 17.059 | 26.12 |
| 6 | 45.4 | 80.94 |
| 13 | 65.3 | 100 |
| Reference product | 56.09 | 85.89 |

TABLE IV
Correspondence between structure and code of the
synthesized products

| Product | Structure |
|---|---|
| 1 | Ac-(Ala)n-Alaol |
| 2 | Ac-(Ala)n-Alaol-Ac |
| 3 | Ac-(Ala)n-Alaol |
| 4 | Dodecyl-(Ala)n-Alaol |
| 5 | Ac-(Ala)n-Alaol |
| 6 | H-(Ala)n-Alaol |
| 7 | H-Ala-Alaol |
| 8 | H-(Ala)₂-Alaol |
| 9 | H-(Ala)₃-Alaol |
| 10 | H-(ala)₄-Alaol |
| 11 | H-(Ala)₅-Alaol |
| 12 | H-(Ala)₆-Alaol |
| 13 | H-(Ala)n-COOH |
| 14 | H-(Ala)₇-Alaol |
| GABA | γ-aminobutyric acid |
| Aib | α-aminoisobutyric acid |
| T.N.T. | Untreated Control |

1. Oligopeptides used as elicitors of the natural defenses of plants against fungal and/or bacterial and/or viral pathogens and/or plant pests by foliage, root or injection application; characterized in that they are obtained by organic or enzymatic synthesis; and that they have the particularity of being homopolymers of protein and/or non-protein amino acids and in that the amino acids constitute sequences of said polymers which are selected for their property to form structures of the spiral type.

2. Oligopeptides, according to claim 1, characterized in that they have the following formula: 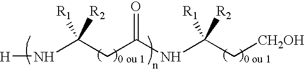
in which R=H, alkyl, substituted alkyl;
according to the L or D configuration of the amino acids used: R₁ and R₂=H, alkyl, substituted or side chain alkyl of natural or non-natural amino acids, protected in the case of functional chains: R₁ and R₂ can be identical;
the N side terminal of the homopolymers being or not acylated;
n being comprised between 3 and 30 in the case of homopolymers obtained in a mixture;
n being comprised between 3 and 20 in the case of pure and characterized homopolymers.

3. Oligopeptides, according to claim 1, characterized in that they have the following formula: 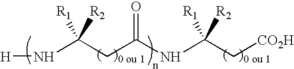
in which R=H, alkyl, substituted alkyl;
according to the L or D configuration of the amino acids used: R₁ and R₂=H, alkyl, substituted or side chain alkyl of natural or non-natural amino acids, protected in the case of functional chains: R₁ and R₂ can be identical;
the N side terminal of the homopolymers being acylated or not;
n being comprised between 3 and 30 in the case of homopolymers obtained in a mixture;
n being comprised between 3 and 20 in the case of pure and characterized homopolymers.

4. Composition characterized in that it comprises at least one oligopeptide obtained according to at least one of the preceding claims and comprising at least one amino acid of the protein and/or non-protein type.

5. Composition according to claim 4, characterized in that the oligopeptides used are incorporated in a vehicle used in agriculture of the wetting and penetrating type.

6. Composition according to claim 5, characterized in that it is present in liquid form, particularly aqueous solution.

7. Composition according to claim 5, characterized in that it is present in the solid form, particularly powder, granules or seed cladding.

8. Utilization of oligopeptides, as defined in claim 1, characterized in that they have the effect of reducing, when they are applied:
in cereals, particularly wheat, corn and rice, the attack of oidiums, septorioses, molds, fusarioses, pyricularioses and bacterial and viral maladies;
in fruit trees, particularly pear trees and apple trees, the attack of oidiums, tavelures, moniloses, bacterial and viral maladies such as “Sharka”;
in grape, the attack of oidium, of mildew, of Botrytis, of maladies of the wood, of telluric and viral maladies such as “Short-Setting”;
in lawns and in horticulture, the attack of pythiaces, mushrooms with sclerotes, fusarioses, oidiums, bacterial and viral maladies;
in oil producers, particularly soy, sunflower, melon, carrot, cauliflower and potato, the attack of oidiums, mildews, phythiaces (Phytophtora, Pythium), mushrooms with sclerotes (Rhizoctonia, Sclerotinia, Pyrenocheta), vascular mushrooms (Fusarium, Verticillium), bacterial and viral maladies.
